(19) United States
(12) Patent Application Publication
(10) Pub. No.: US 2025/0090366 A1
(43) Pub. Date: Mar. 20, 2025

(54) OSTOMY GAS RELEASE CHAMBER AND OTHER WASTE MANAGEMENT EXTENSIONS

(71) Applicant: BOSTON SCIENTIFIC SCIMED, INC., Maple Grove, MN (US)

(72) Inventors: Colin BUCKLEY, Brookline, MA (US); Allison KUMARASENA, Wellesley, MA (US); Rahul PRABHU, Brighton, MA (US); Laura Emily SABBAN, Plainville, MA (US); Kurt Nicholas ROBAKIEWICZ, Upton, MA (US); Cristina ROMANY, Orlando, FL (US); Lisa Margaret SANGREE, Boston, MA (US); Julia VEITCH, Waltham, MA (US)

(21) Appl. No.: 18/800,593

(22) Filed: Aug. 12, 2024

Related U.S. Application Data
(60) Provisional application No. 63/582,684, filed on Sep. 14, 2023.

(51) Int. Cl. A61F 5/445; A61F 5/44; A61F 5/441

(52) U.S. Cl. CPC A61F 5/445; A61F 5/4405; A61F 5/441; A61F 2005/4415

(57) ABSTRACT

An ostomy bag device includes a bag with first and second chambers communicating via a first opening and a pressure sensitive interface. The bag includes a second opening interfacing with a stoma exit and receiving waste in the first chamber, a third opening expelling the waste from the first chamber, and a fourth opening releasing gas from the second chamber. The interface transitions from a first state in which the interface prevents a gas flow through the first opening from the first to second chambers and a second state in which the interface allows the flow. A first pressure differential in the first chamber relative to the second chamber causes the interface to assume the first state preventing the flow and a second pressure differential in the first chamber relative to the second chamber higher than the first differential causes the interface to assume the second state allowing the flow.

### PRIORITY CLAIM

The present disclosure claims priority to U.S. Provisional Patent Application Ser. No. 63/582,684 filed Sep. 14, 2023; the disclosure of which is incorporated herewith by reference.

### FIELD

The present disclosure relates to ostomy bag devices for post-procedural ostomy management.

### BACKGROUND

An ostomy refers to a surgical procedure in which an opening, or stoma, is created in the abdomen to provide a path for human waste or a stool (e.g., a liquid waste, a fluid, a solid waste, and/or a gas waste) to be removed from the body. Sections of the gastrointestinal tract are bypassed, and the waste typically produced and transported through the body is excreted through the stoma. Traditionally, ostomies are managed through technology such as an ostomy bag, which the patient must regularly vacate and replace.

One of the greatest issues patients face with ostomies are the social pressures, including gas build up and odor. It is possible for the ostomy bag to fill up with gas and explode if the pressure of the gas is not released. Another concern is the odor the ostomy can produce, over which the patient has no control. In addition, it can be difficult to dispose of the waste from the ostomy bag.

### SUMMARY

The present disclosure relates to an ostomy bag device. The bag device includes a bag comprising a first chamber and a second chamber in communication via a first opening in a divider between the first chamber and the second chamber. The bag includes a second opening configured to interface with a stoma exit and receiving waste in the first chamber, a third opening configured to expel the waste from the first chamber, and a fourth opening configured to release gas from the second chamber.

The bag device also includes a first pressure sensitive interface located at the first opening configured to transition from a first state in which the first pressure sensitive interface prevents a flow of gas through the first opening from the first chamber to the second chamber and a second state in which the first pressure sensitive interface allows the flow of gas through the first opening from the first chamber to the second chamber. A first pressure differential in the first chamber relative to the second chamber causes the first pressure sensitive interface to assume the first state preventing the flow of gas and a second pressure differential in the first chamber relative to the second chamber higher than the first pressure differential causes the first pressure sensitive interface to assume the second state allowing the flow of gas.

In an embodiment, the first pressure sensitive interface comprises a valve configured, in the first state, to block the first opening and, in the second state, to at least partially unblock the first opening.

In an embodiment, the valve comprises a spring attached to a first end of the valve and a second end of the valve, the first end of the valve being attached to an interior of the second chamber, expansion of the spring causing the second end of the valve to extend at least partially through the first opening to block the flow of gas through the first opening, contraction of the spring causing the second end to withdraw from the first opening to allow the flow of gas through the first opening.

In an embodiment, the first pressure differential allows the spring to expand and the second pressure differential forces the spring to contract.

In an embodiment, the valve comprises a relief valve.

In an embodiment, the first pressure sensitive interface comprises a semi-permeable membrane includes a seal that breaks in the first state and closes in the second state.

In an embodiment, the bag device further includes a second pressure sensitive interface including a release valve located at the fourth opening configured to transition from the first state in which the release valve prevents a flow of gas through the fourth opening from the second chamber to an environment exterior to the bag and the second state in which the release valve allows the flow of gas through the fourth opening from the second chamber to the environment.

In an embodiment, the release valve comprises an actuator to manually open the release valve to allow the flow of gas through the fourth opening or manually close the release valve to prevent the flow of gas through the fourth opening.

In an embodiment, the second pressure sensitive interface comprises an external element for notifying a patient when gas is present in the second chamber.

In an embodiment, the release valve comprises an odor control mechanism for the flow of gas through the fourth opening.

In addition, the present disclosure relates to an ostomy bag device which includes a bag including a first chamber into which waste can be received. The bag device also includes an exit flap configured to transition between a withdrawn state in which the waste is prohibited from passing through the exit flap and an extended state in which the waste is allowed to pass through the exit flap. The exit flap is naturally biased to transition into or remain in the withdrawn state.

In an embodiment, the exit flap is further configured to transition into a partially extended state in which the waste is prohibited from passing through the exit flap and an internal volume of the bag is increased to include both the first chamber and a volume within the exit flap.

In an embodiment, the exit flap is transitioned into the extended state by an external force pulling distally on the exit flap.

In an embodiment, the bag device further includes a button on an exterior of the bag configured to manually lock or unlock the exit flap. The exit flap is configured to be locked in the withdrawn state, the extended state, or the partially extended state.

In an embodiment, in the extended state, the exit flap assumes a curved geometry and wherein, in the withdrawn state or the partially extended state, the exit flap assumes a flat geometry to facilitate coiling the exit flap.

In an embodiment, the exit flap comprises a distal locking mechanism comprising a zipper or a sliding lock.

In addition, the present disclosure relates to an ostomy bag device which includes a bag including a chamber configured to receive waste. The bag includes a first exit opening sized and shaped for expelling the waste and a second exit opening smaller than the first exit opening sized and shaped for expelling fluid. The bag device includes a tubing channel extending from the second exit opening configured to expel the fluid.

In an embodiment, a distal end of the tubing channel comprises a valve configured to manually expel the fluid from the tubing channel.

In an embodiment, a flow rate of the valve is adjustable by a patient.

In an embodiment, the bag device further includes a clip configured to hold a distal end of the tubing channel when not in use.

In addition, the present disclosure relates to a method which includes attaching to a stoma exit an ostomy bag device comprising a bag comprising a first chamber and a second chamber in communication via a first opening in a divider between the first chamber and the second chamber, the bag including a second opening configured to interface with the stoma exit and receiving waste in the first chamber, a third opening configured to expel the waste from the first chamber, and a fourth opening configured to release gas from the second chamber, the ostomy bag device comprising a first pressure sensitive interface located at the first opening configured to transition from a first state in which the first pressure sensitive interface prevents a flow of gas through the first opening from the first chamber to the second chamber and a second state in which the first pressure sensitive interface allows the flow of gas through the first opening from the first chamber to the second chamber, the ostomy bag device comprising a second pressure sensitive interface including a release valve located at the fourth opening configured to transition from the first state in which the release valve prevents a flow of gas through the fourth opening from the second chamber to an environment exterior to the bag and the second state in which the release valve allows the flow of gas through the fourth opening from the second chamber to the environment wherein the release valve comprises an actuator to manually open the release valve to allow the flow of gas through the fourth opening or manually close the release valve to prevent the flow of gas through the fourth opening; and opening the release valve to allow the flow of gas through the fourth opening.

In an embodiment, a first pressure differential in the first chamber relative to the second chamber causes the first pressure sensitive interface to assume the first state preventing the flow of gas and a second pressure differential in the first chamber relative to the second chamber higher than the first pressure differential causes the first pressure sensitive interface to assume the second state allowing the flow of gas.

In an embodiment, the first pressure sensitive interface comprises a valve configured, in the first state, to block the first opening and, in the second state, to at least partially unblock the first opening.

In an embodiment, the valve comprises a spring attached to a first end of the valve and a second end of the valve, the first end of the valve being attached to an interior of the second chamber, expansion of the spring causing the second end of the valve to extend at least partially through the first opening to block the flow of gas through the first opening, contraction of the spring causing the second end to withdraw from the first opening to allow the flow of gas through the first opening.

In an embodiment, the first pressure differential allows the spring to expand and the second pressure differential forces the spring to contract.

### BRIEF DESCRIPTION

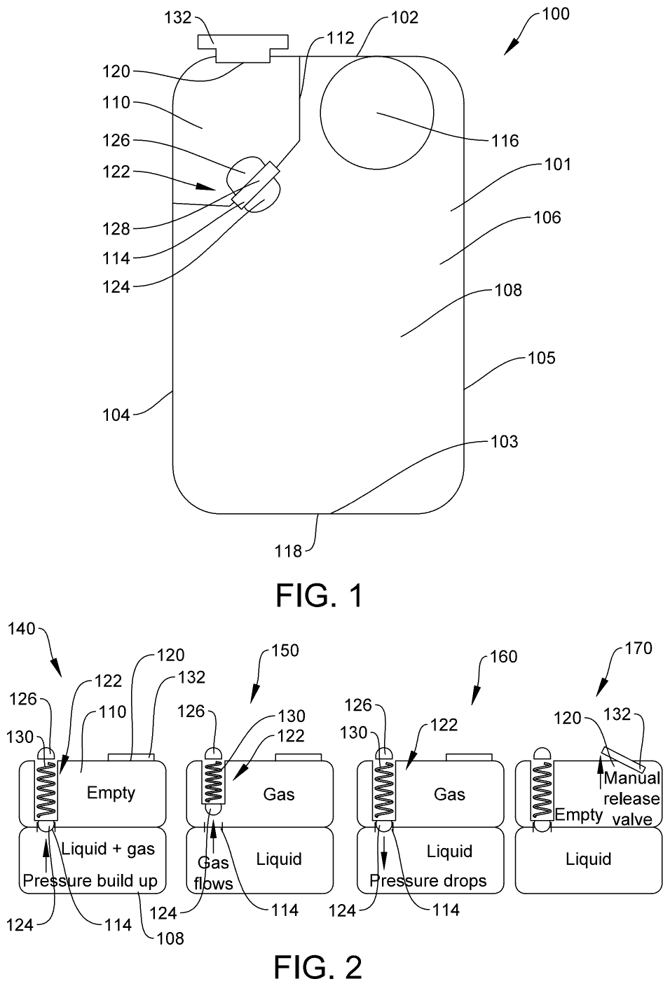

FIG. 1 shows a diagram of an ostomy bag device comprising a bag including a secondary chamber in communication with a primary chamber via a pressure sensitive interface for relieving gaseous pressure in the primary chamber.

FIG. 2 shows gas release operations of the ostomy bag device described in FIG. 1.

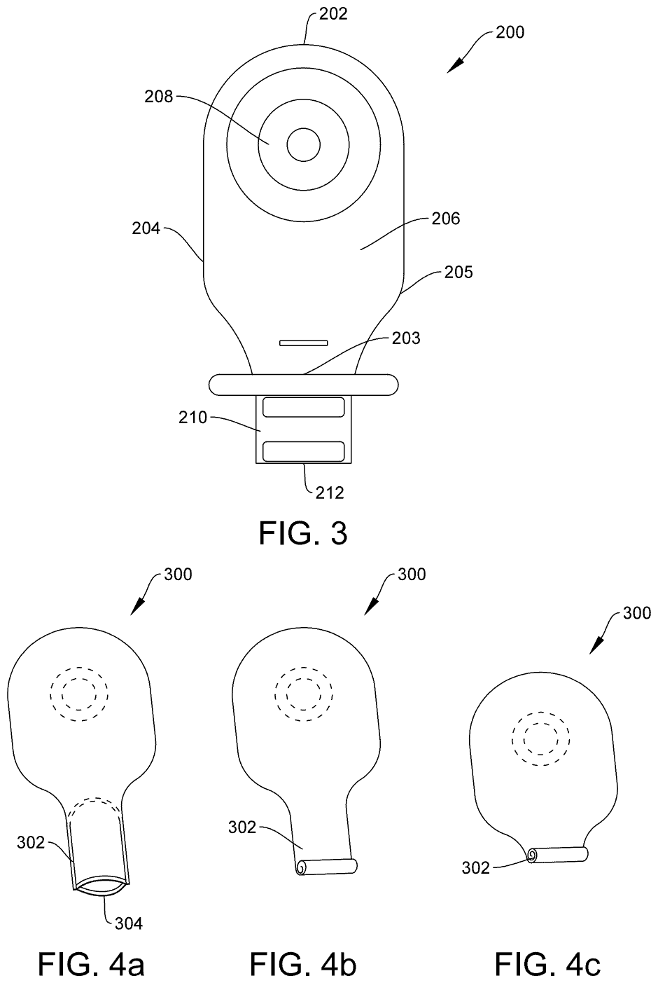

FIG. 3 shows an ostomy bag according to conventional designs.

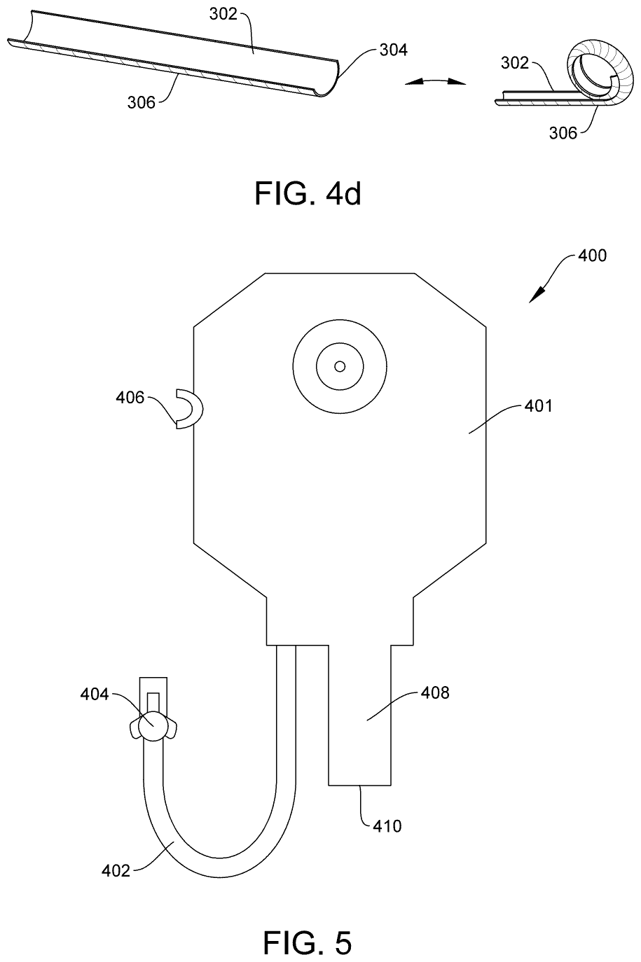

FIG. 4a shows an ostomy bag device including an extendable and retractable exit flap according to another exemplary embodiment of the present disclosure.

FIG. 4b shows the ostomy bag device of FIG. 4a with the exit flap in a partially extended state.

FIG. 4c shows the ostomy bag device of FIG. 4a with the exit flap in a fully retracted state.

FIG. 4d shows the geometry of the exit flap of the ostomy bag device of FIG. 4a.

FIG. 5 shows an ostomy bag device including a bag for receiving the waste and a tube providing a path for fluid expulsion from the bag.

### DETAILED DESCRIPTION

The present disclosure may be further understood with reference to the following description and the appended drawings, wherein like elements are referred to with the same reference numerals. The present disclosure relates to ostomy bag devices comprising one or more features for enhancing the usability of the ostomy bag and alleviating the social pressures associated with post-procedural ostomy management.

There are several types of ostomy procedures including a colostomy, an ileostomy and a urostomy. In a colostomy, portions of the large intestine (e.g., colon and/or rectum) are removed and the remaining tissue is brought through an opening in the abdomen to form the stoma. In an ilcostomy, the ileum (end of the small intestine) is brought through the abdomen to form the stoma. After these procedures are performed, the waste can then be passed through the stoma, e.g., into an ostomy bag. In a urostomy, a portion of the small intestine is detached from the rest of the small intestine and brought through the abdomen to form a stoma. This detached portion of the small intestine is coupled to the ureters to allow for the passage of urine away from the bladder and out of the stoma to an ostomy bag. Some ostomy procedures can be performed laparoscopically using small incisions while others are performed as open surgeries using a larger incision. Some ostomy procedures are permanent while others are reversible. Different procedures can require different durations for which the ostomy bag must be used.

The stoma may be flat against the skin or protrude a small distance out the abdomen. The pouching system for receiving the waste via the stoma can comprise a one-piece ostomy bag attaching directly to the abdomen around the stoma to form a seal or may comprise a two-piece system including a flange attached to the abdomen with the ostomy bag attached to the flange to form the seal. The ostomy bag has a first opening (e.g., an entrance opening) on or adjacent to its top side for interfacing with the stoma and receiving the waste and a second opening (e.g., a waste exit) on or adjacent to its bottom side for disposing of the waste. The ostomy bag may include a distal extension (e.g., an exit flap) with a distal end comprising the waste exit that can be rolled up and sealed, e.g., using Velcro, when not in use.

The ostomy bag can be externally supported at the abdomen, e.g., by adhesive or by using a belt to which the ostomy bag attaches on its top side. Ostomy bags come in a variety of different sizes, shapes and styles. For example, some ostomy bags lie flat against the skin so that the bag can be worn discreetly for daily use. Some ostomy bags are reusable while others are single use. Some ostomy bags are vented to allow for the release of gas built up in the pouch.

FIG. 3 shows an ostomy bag 200 according to conventional designs. The bag 200 includes a first side 202 (e.g., a top side when the bag 200 is oriented in its intended manner on the body of the patient); a second side 203 (e.g., a bottom side); a third side 204 (e.g., a left side—from the perspective of the patient wearing the bag 200); and a fourth side 205 (e.g., a right side). The first side 202 may be referred to as a proximal side and the second side 203 may be referred to as a distal side. The bag 200 further comprises a fifth side 206 (e.g., a back side—facing inward toward the patient wearing the bag) and a sixth side (obscured in FIG. 3 and not labeled) (e.g., a front side—facing outward from the perspective of the patient wearing the bag). In this example, the bag 200 has a rounded top and a tapered bottom. The bag 200 may have a depth (e.g., a distance between the fifth side 206 (back) and sixth side (front)) sufficiently small so that the bag 200 can be worn discreetly under a patient's clothing. The bag 200 is generally formed of a pliable material that permits the bag to expand in volume when the bag 200 fills with the waste and to contract when the bag 200 is emptied.

The sides of the bag 200 define an interior volume configured to store the waste prior to the expulsion of the waste from the bag 200. The bag 200 has an entrance opening 208 in the fifth side 206 (e.g., the back side) adjacent to the first side 202 (e.g., the top side) through which the waste is received via the stoma to pass into the bag 200. The bag 200 further has an exit opening 212 (e.g., a waste exit) at a distal end of an exit flap 210 extending from the second side 203 (e.g., the bottom side) of the bag 200 through which the waste may be expelled from the bag 200. The exit flap 210 can be rolled up and sealed to the second side 203 when not in use.

While generally effective, conventional ostomy bags leave room for improvement. One of the major patient complaints with regard to existing ostomy bags is the buildup of gas, which has the potential to expand and weaken the seal to the patient at the entrance opening 208. In some cases, the gaseous pressure may cause the bag to rupture.

Ostomy bag devices according to various exemplary embodiments include a second chamber (e.g., a secondary chamber) for relieving gaseous pressure from a first chamber (e.g., a primary chamber) to prevent failure of the ostomy bag. A pressure sensitive interface between the primary and secondary chambers allows gas to pass from the primary chamber to the secondary chamber when the pressure in the primary chamber is sufficiently high, e.g., when a pressure differential between the primary chamber and the secondary chamber is greater than a predetermined differential at which the interface is opened by the pressure in the primary chamber. The pressure sensitive interface is configured to prevent gas from passing from the primary chamber to the secondary chamber when the pressure in the primary chamber is low (e.g., when the pressure differential between the primary chamber and the secondary chamber is less than the predetermined threshold below which the interface remains closed).

In some embodiments, the pressure sensitive interface between the primary and secondary chambers is a valve (e.g., a pop valve or a relief valve). This valve is typically closed (e.g., when the pressure in the primary chamber is relatively low). When gas in the primary chamber expands to a certain volume and pressure, the valve inverts to allow gas to flow into the secondary chamber. When the pressures in the respective chambers have equalized (e.g., when the differential between the primary and secondary chambers has dropped below the predetermined threshold), the valve closes again.

FIG. 1 shows an ostomy bag device 100 comprising a bag 101 including a secondary chamber 110 in communication with a primary chamber 108 via a pressure sensitive interface for relieving gaseous pressure in the primary chamber 108. The bag 101 includes a first side 102 (e.g., a top side when the bag 101 is oriented in its intended manner on the body of the patient body); a second side 103 (e.g., a bottom side); a third side 104 (e.g., a left side—from the perspective of the patient wearing the bag 101); and a fourth side 105 (e.g., a right side). The bag 101 further comprises a fifth side 106 (e.g., a back side—facing inward toward the patient wearing the bag 101) and a sixth side (obscured in FIG. 1 and not labeled) (e.g., a front side—facing outward from the perspective of the patient wearing the bag 101).

The ostomy bag device 100 of FIG. 1 is shown in simplified form and is representative of a variety of different types of ostomy bags, e.g., a colostomy bag or ilcostomy bag, and can comprise various sizes and shapes depending on patient needs. Thus, it should be understood that the sides 102-105 may not be strictly defined as shown in FIG. 1 (e.g., may not form a substantially rectangular shape) and are demarcated as shown for ease of explanation. In one example, the bag 101 has a rounded top and a tapered bottom, as shown, e.g., in FIG. 3.

The bag 101 may be formed of a pliable material (e.g., plastic or polymer) that permits the bag 101 to expand in volume when the bag 101 fills with the waste and to collapse when the bag 101 is emptied. The sides 102-105 of the bag 101 define an interior volume. The interior volume is, for example, at a minimum when the bag 101 is empty of the waste and can expand to a maximum when the bag 101 is filled with the waste.

According to the present embodiments, the interior volume of the bag 101 is divided into the primary chamber 108 and the secondary chamber 110 by a divider 112. The divider 112 can be formed, for example, of the same material as the bag 101 (e.g., plastic or polymer). The primary chamber 108 comprises a first portion of the interior volume (i.e., a first volume) and the secondary chamber 110 comprises a second portion of the interior volume (i.e., a second volume). The secondary chamber 110 in this embodiment is located near or at the top of the bag 101 (i.e., the first side 102).

As shown in FIG. 1, the divider 112 extends from a location on the first side 102 and curves toward the left side (e.g., the third side 104) to define the second volume of the secondary chamber 110 and to seal the secondary chamber 110 off from the primary chamber 108. However, the secondary chamber 110 may be defined in any of a variety of locations within the interior volume of the bag 101 according to the considerations described below (e.g., so that a release valve 132 for the secondary chamber 110 is easily accessible by the patient and the non-gaseous contents of the bag 101 do not interfere with the release valve 132). The primary chamber 108 of this embodiment comprises the majority of the internal volume of the bag 101 extending, e.g., from a portion of the bag 101 adjacent to the top to an entirety of the bottom of the bag 101.

The primary chamber 108 and the secondary chamber 110 are in communication via a first opening in the divider 112 (e.g., a divider opening 114). To be described further below, in the example of FIGS. 1-2, a pressure sensitive interface comprising a valve 122 (e.g., a spring valve) is positioned within the bag 101 so that a portion of the valve 122 can cover the divider opening 114 and isolate the primary chamber 108 from the secondary chamber 110 when the valve 122 is in the closed state. The valve 122 can allow gas to pass from the primary chamber 108 to the secondary chamber 110 across the divider opening 114 when the valve 122 is in the open state.

The bag 101 has a second opening (e.g., an entrance opening 116) in the primary chamber 108 through which the waste is received into the primary chamber 108 via the stoma exit. The entrance opening 116 is preferably located on the bag 101 adjacent to the first side 102 (e.g., near the top of the bag 101) so that the waste enters the primary chamber 108 and falls toward the second side 103 due to gravity. This permits the primary chamber 108 to be filled without the received waste interfering with the entrance opening 116. The entrance opening 116 can interface with the stoma exit according to existing art. The primary chamber 108 can be emptied via a third opening (e.g., an exit opening 118) (not shown in detail in FIG. 1) on the second side 103 of the ostomy bag device 100, to be described in further detail below.

The exit opening 118 may, for example, be located at the end of an exit flap (not shown), to be described in further detail below. The bag 101 of this embodiment has a fourth opening (e.g., a release opening 120) permitting the release of gas from the secondary chamber 110. To be described further below, in the example of FIGS. 1-2, positioned across the release opening 120 is the release valve 132 that is manually actuatable so that the patient can expel gas from the secondary chamber 110 as desired, e.g., in a discreet location.

The primary chamber 108 and the secondary chamber 110 are in communication across the divider opening 114 only via the valve 122. In this example, the valve 122 comprises a pressure pop valve including a first end 124 extending at least partially into the divider opening 114 and/or into the primary chamber 108 with a second end 126 of the valve 122 extending into the secondary chamber 110, and a body 128 of the valve 122 extending between the first end 124 and the second end 126. In the example shown in FIG. 2, the body 128 comprises a spring 130 that contracts when pressure from the primary chamber 108 urges the first end 124 out of the divider opening 114 and into the secondary chamber 110 permitting gas to flow through the divider opening 114.

FIG. 2 shows one example of a valve system for use with devices according to the disclosed embodiments. The design functions with a spring 130 in the secondary chamber 110 (e.g., a gas release chamber). When pressure builds up in the primary chamber 108, the spring 130 contracts to permit gas to flow into the secondary chamber 110. After the pressure has dropped in the primary chamber 108, or when the pressure differential between the primary chamber 108 and the secondary chamber 110 has dropped below a predetermined level, the spring 130 expands back to its original position resealing the valve 122.

According to another aspect of these exemplary embodiments, the secondary chamber 110 may have an opening to the exterior of the bag 101 with a pressure sensitive interface comprising a release valve that can be manually actuated by the patient when desired, e.g., when the patient is in a discreet location. The release valve may include means for odor mitigation such as, e.g., an activated carbon charcoal filter. In some embodiments, the patient will be notified when the pressure in the secondary chamber 110 has increased to the predetermined level.

In one example, a notification element on the exterior of the pressure sensitive interface triggers (e.g., pops out of the release valve) to indicate that the patient may want to evacuate the secondary chamber 110. The notification element may be triggered, for example, when a relatively low pressure is applied to the pressure sensitive interface by the secondary chamber 110, e.g., to indicate that the pressure valve between the primary and secondary chambers 108, 110 has allowed gas to flow from the primary chamber 108 into the secondary chamber 110, or when a higher pressure is applied to the pressure sensitive interface by the secondary chamber 110 (e.g., to indicate that the secondary chamber 110 is at or near a maximum desired pressure and should be vacated urgently). Alternatively, the patient may physically notice inflation of the secondary chamber 110 (e.g., by sight or by sound) and thus be notified that it may be desirable to evacuate the secondary chamber 110.

Referring back to FIG. 1, the secondary chamber 110 can be located in the bag 101 so that the release valve 132 is easily accessible by the patient to release gas from the secondary chamber 110. Thus, the secondary chamber 110 is preferably located toward the top of the bag 101 (e.g., the first side 102) and outward-facing (e.g., toward the front (sixth side)) so that the release valve 132 is visible to the patient and so that, even if the patient is lying down, the gas will rise toward the secondary chamber 110. Further, this location for the secondary chamber 110 will provide ease of release of the secondary chamber 110 in multiple patient positions, such as sitting, laying down, or standing upright.

FIG. 2 shows gas release operations 140-170 of the ostomy bag device 100 described in FIG. 1. The operations 140-170 are shown with regard to the valve 122 located adjacent to the divider opening 114 between the primary chamber 108 and the secondary chamber 110 and the release valve 132 located adjacent to the release opening 120 in the secondary chamber 110. In this example, the valve 122 comprises a spring 130 disposed between the first end 124 and the second end 126. When the valve 122 is closed, e.g., preventing the flow of gas across the divider opening 114, the spring 130 is in an expanded state so that the first end 124 extends at least partially into the divider opening 114 to block the divider opening 114, as shown in the operation 140.

The second end 126 is fixed to the interior of the secondary chamber 110 so that, when pressure is applied to the first end 124 by the contents of the primary chamber 108, the spring 130 is compressed pushing the first end 124 into the secondary chamber 110 and unblocking the divider opening 114 so that gas may flow across the divider opening 114 into the secondary chamber 110, as shown in the operation 150. The fix point for the second end 126 in the secondary chamber 110 remains in a fixed position relative to the divider opening 114 so that, when the pressures across the primary and secondary chambers 108, 110, respectively, has equalized, the spring 130 will expand driving the first end 124 toward the divider opening 114 so that the first end 124 reenters the divider opening 114, as shown in the operation 160.

It should be understood that the valve 122 (e.g., a pop valve) shown in FIG. 2 is exemplary only. In another embodiment, the pressure sensitive interface may comprise a relief valve or any other suitable structure as would be understood by those skilled in the art. In still another embodiment, the pressure sensitive interface between the primary and secondary chambers 108, 110 includes a semi-permeable membrane including, for example, a seal that opens when a pressure differential between the primary and secondary chambers 108, 110 exceeds a threshold level and which then is applied and which then re-seals when the pressure differential is reduced below the threshold level.

At this point the patient may become aware of a buildup of gas in the bag 101 and use the release valve 132 attached to the top of the secondary chamber 110 to open the release opening 120 to manually release the gas from the secondary chamber 110, as shown in the operation 170. Additionally, the release valve 132 may have an odor control system (e.g., an activated carbon charcoal filter). The benefits to this valve system include allowing the patient to expel gas in a more discreet environment. After the patient releases the gas from the secondary chamber 110, the release valve 132 may be re-closed so the system is ready to be used again.

If the release valve 132 has a notification element (e.g., an element that pops up under pressure), this notification element may be re-set (e.g., popped back down) so that the patient may be notified again when the secondary chamber 110 fills at a later time. Additionally, the release valve 132 can include an optional locking mechanism to prevent accidental opening of the release valve 132 (e.g., at an undesirable time). This system may reduce the occurrence of ostomy bag ruptures due to gas buildup while allowing the patient a more manageable means to control odors associated with the bag.

Another issue with existing ostomy bags is the difficulty of disposing of the waste from the ostomy bag, which may be time-consuming, inconvenient and unpleasant. Traditional ostomy bags have a waste exit formed in a flap towards the bottom of the bag for emptying the waste. Referring, for example, to the exit flap 210 and the exit opening 212 of FIG. 3, these openings are typically no more than a few inches in length and are often manually rolled up and sealed (e.g., via a Velcro seal) when the patient is performing day-to-day tasks. To empty the bag, a patient must sit on or adjacent to a toilet and empty the bag through the opening that this exit provides. Due to the short length of the exit flap, there is a risk of the waste spilling onto the patient's garments or self if the patient does not carefully direct the waste away from them towards the toilet bowl.

In another aspect of these exemplary embodiments, the ostomy bag includes an extended exit flap for dispelling the waste from the bag further away from the patient. In one embodiment, the extended exit flap may be rolled up on the exterior of the bag when not in use and unrolled when it is desired to empty the bag. In this embodiment, the exit flap of the ostomy bag may be extended in length relative to existing bags so that the waste can be dispelled from the bag further from the patient. There are a variety of ways to manage this extended length so that it remains unobtrusive with minimal interference to the daily ostomy management. In one design, the extra length may be rolled up and Velcro-sealed, similar to typical ostomy bag designs.

Other embodiments include means to extend and retract the exit flap of the ostomy bag to set a length of the patient's choosing. The length of the exit flap according to such an embodiment may be adjusted by the patient and locked at a desired length. The mechanism for achieving this may be similar to the auto-winding or coiling mechanisms employed in tape measures in which a length of material can assume a rigid state when extended and can become flexible in order to roll up. In a fully extended and rigid state, the shape of the exit flap can enable waste removal. In a fully retracted state, the ostomy bag assumes a minimum volume for discreet patient use. In a partially extended state, the interior volume of the bag is increased relative to the fully rolled up state to enable an increased waste storage volume. It should be understood that, in the fully rolled up or partially extended state, the geometry of the exit flap will prohibit the emptying of waste and only in the fully extended state can waste be removed.

The patient extends the length of the exit flap by pulling on the distal portion of the exit flap until a desired length is pulled out and then locks this length in place via a locking button located, e.g., on the exterior of the bag at its distal end adjacent to a proximal end of the exit flap. Like a tape measure, once the desired length has been locked into place, the extended length will become rigid to better allow the patient to expel the waste from the bag. In the rigid state, the geometry of the exit flap assumes a curved cross section that further enable waste removal. In particular, a first surface of the exit flap (e.g., the surface facing proximally toward the patient) can be configured for the auto-winding feature and can assume the curved cross section when made rigid and, when the exit flap is made flexible, assumes a flat cross section that can be rolled up. A second surface of the exit flap (e.g., the surface facing distally away from the patient) can be flexible so that, when the cross section of the first surface is curved, the second surface is pulled tight from the sides of the curved shape to define the channel of the exit flap and, when the cross section of the first surface is flat, the second surface is brought adjacent to the first surface.

FIG. 4a shows an ostomy bag device 300 including an extendable and retractable exit flap 302 according to another exemplary embodiment of the present disclosure. The exit flap 302 can transition between a retracted state, a partially extended state and an extended state. The exit flap 302 comprises a waste exit 304 at its distal end. In the example of FIG. 4a, the exit flap 302 is in the fully extended state. The ostomy bag device 300 further includes a lock (not shown) configured to lock the exit flap 302 at a current length and unlock the exit flap 302 to adjust its length.

FIG. 4b shows the ostomy bag device 300 of FIG. 4a with the exit flap 302 in a partially extended state. To transition the exit flap 302 away from the fully extended state, the patient can unlock the exit flap 302. The patient can apply a distal force to the exit flap 302 to set its length and lock the exit flap 302 at this length.

FIG. 4c shows the ostomy bag device 300 of FIG. 4a with the exit flap 302 in a fully retracted state. Similar to the above, the patient can unlock the exit flap 302 and allow it to automatically coil. The patient can lock the exit flap 302 in the fully retracted state, however, this is not required as the exit flap 302 is naturally biased to assume the fully retracted state.

FIG. 4d shows the geometry of the exit flap 302 of the ostomy bag device 300 of FIG. 4a. In 310, the exit flap 302 is fully extended and a surface 306 of the exit flap 302 is curved. In 320, the exit flap 302 is partially rolled up and the surface 306 of the exit flap 302 is straightened to facilitate the coiling.

It should be understood that the extended waste exit according to the present embodiments may be integrated into any ostomy bag device described in the present disclosure (e.g., the two-chamber ostomy bag device described above) or other types of ostomy bags (e.g., existing one-chamber ostomy bags).

Another issue with existing ostomy bags is a risk of leakage from the waste exit. As previously mentioned, the waste exit of a typical ostomy bag is sealed via a Velcro-Seal. This may present a risk of leakage if the strength of the seal is compromised due to waste particulate trapped between the Velcro fibers.

In another aspect of these exemplary embodiments, alternate locking mechanisms may be used for the waste exit to reduce the risk of leakage. Alternate locking mechanisms for the waste exit can include a zipper design, sliding lock design, or a combination of mechanisms for double protection. In the sliding lock design, the patient would slide a lock across the opening of the waste exit to form a seal.

It should be understood that the alternate locking mechanism for the waste exit according to the present embodiments can be integrated into any ostomy bag device described in the present disclosure, e.g., having an extended waste exit, or other types of ostomy bags, e.g., having a waste exit typical of existing ostomy bags.

Another issue with existing ostomy bags is that most bags contain only a single exit (e.g., the waste exit) that is short and wide to allow for easy passage of the waste from the bag. However, this design is less effective for dispelling fluid in a controlled manner. In the case of most ostomy procedures, especially ileostomies, the resulting bodily output channeled into an ostomy bag is mostly fluid.

In another aspect of these exemplary embodiments, the ostomy bag includes an added tubing channel allowing the patient to dispel fluid from the ostomy bag out of reach of any clothing and reduce the need to have to sit while emptying their ostomy bag. A valve locking mechanism allows the patient to dispel fluid through the channel at a rate of their choosing. A clip-on lock located near the top of the bag may be used to manage the length of the channel when not in use.

FIG. 5 shows an ostomy bag device 400 according to a further exemplary embodiment of the present disclosure. The ostomy bag device 400 may include a bag 401 configured to receive the waste and a tube 402 providing a path for fluid expulsion from the bag 401. The tube 402 provides a second path for the expulsion of the waste in addition to a first path comprising an exit flap 408 and a waste exit 410 on its distal end. The waste exit 410 may be used to expel the solid waste while the tube 402 may be used to expel the liquid waste.

The exit flap 408 and the tube 402 may both extend from a bottom of the bag 401. The length of the tube 402 may be selected for ease of use by a patient. The tube 402 comprises a valve 404 at its distal end for the patient to control the expulsion of fluid from the bag 401. The valve 404 can be actuated by the patient to allow the expulsion of fluid at varying flow rates. The ostomy bag device 400 further includes a clip 406 on its exterior for holding the tube 402 when not in use.

It will be appreciated by those skilled in the art that changes may be made to the embodiments described above without departing from the inventive concept thereof. It should further be appreciated that structural features and methods associated with one of the embodiments can be incorporated into other embodiments. It is understood, therefore, that this invention is not limited to the particular embodiment disclosed, but rather modifications are also covered within the scope of the present invention as defined by the appended claims.

1-16. (canceled)

17. An ostomy bag device, comprising:
a bag comprising a first chamber and a second chamber in communication via a first opening in a divider between the first chamber and the second chamber, the bag including a second opening configured to interface with a stoma exit and receiving waste in the first chamber, a third opening configured to expel the waste from the first chamber, and a fourth opening configured to release gas from the second chamber; and
a first pressure sensitive interface located at the first opening configured to transition from a first state in which the first pressure sensitive interface prevents a flow of gas through the first opening from the first chamber to the second chamber and a second state in which the first pressure sensitive interface allows the flow of gas through the first opening from the first chamber to the second chamber,
wherein a first pressure differential in the first chamber relative to the second chamber causes the first pressure sensitive interface to assume the first state preventing the flow of gas and a second pressure differential in the first chamber relative to the second chamber higher than the first pressure differential causes the first pressure sensitive interface to assume the second state allowing the flow of gas.

18. The ostomy bag device of claim 17, wherein the first pressure sensitive interface comprises a valve configured, in the first state, to block the first opening and, in the second state, to at least partially unblock the first opening.

19. The ostomy bag device of claim 18, wherein the valve comprises a spring attached to a first end of the valve and a second end of the valve, the first end of the valve being attached to an interior of the second chamber, expansion of the spring causing the second end of the valve to extend at least partially through the first opening to block the flow of gas through the first opening, contraction of the spring causing the second end to withdraw from the first opening to allow the flow of gas through the first opening.

20. The ostomy bag device of claim 19, wherein the first pressure differential allows the spring to expand and the second pressure differential forces the spring to contract.

21. The ostomy bag device of claim 18, wherein the valve comprises a relief valve.

22. The ostomy bag device of claim 17, wherein the first pressure sensitive interface comprises a semi-permeable membrane includes a seal that breaks in the first state and closes in the second state.

23. The ostomy bag device of claim 17, further comprising:
a second pressure sensitive interface including a release valve located at the fourth opening configured to transition from the first state in which the release valve prevents a flow of gas through the fourth opening from the second chamber to an environment exterior to the bag and the second state in which the release valve allows the flow of gas through the fourth opening from the second chamber to the environment.

24. The ostomy bag device of claim 23, wherein the release valve comprises an actuator to manually open the release valve to allow the flow of gas through the fourth opening or manually close the release valve to prevent the flow of gas through the fourth opening.

25. The ostomy bag device of claim 23, wherein the second pressure sensitive interface comprises an external element for notifying a patient when gas is present in the second chamber.

26. The ostomy bag device of claim 23, wherein the release valve comprises an odor control mechanism for the flow of gas through the fourth opening.

27. An ostomy bag device, comprising:
a bag including a first chamber into which waste can be received; and
an exit flap configured to transition between a withdrawn state in which the waste is prohibited from passing through the exit flap and an extended state in which the waste is allowed to pass through the exit flap,
wherein the exit flap is naturally biased to transition into or remain in the withdrawn state.

28. The ostomy bag device of claim 27, wherein the exit flap is further configured to transition into a partially extended state in which the waste is prohibited from passing through the exit flap and an internal volume of the bag is increased to include both the first chamber and a volume within the exit flap.

29. The ostomy bag device of claim 28, wherein the exit flap is transitioned into the extended state by an external force pulling distally on the exit flap.

30. The ostomy bag device of claim 28, further comprising:
a button on an exterior of the bag configured to manually lock or unlock the exit flap,
wherein the exit flap is configured to be locked in the withdrawn state, the extended state, or the partially extended state.

31. The ostomy bag device of claim 28, wherein, in the extended state, the exit flap assumes a curved geometry and wherein, in the withdrawn state or the partially extended state, the exit flap assumes a flat geometry to facilitate coiling the exit flap.

32. The ostomy bag device of claim 28, wherein the exit flap comprises a distal locking mechanism comprising a zipper or a sliding lock.

33. An ostomy bag device, comprising:
a bag including a chamber configured to receive waste, the bag including a first exit opening sized and shaped for expelling the waste and a second exit opening smaller than the first exit opening sized and shaped for expelling fluid; and
a tubing channel extending from the second exit opening configured to expel the fluid.

34. The ostomy bag device of claim 33, wherein a distal end of the tubing channel comprises a valve configured to manually expel the fluid from the tubing channel.

35. The ostomy bag device of claim 34, wherein a flow rate of the valve is adjustable by a patient.

36. The ostomy bag device of claim 33, further comprising:
a clip configured to hold a distal end of the tubing channel when not in use.

37. A method, comprising:
attaching to a stoma exit an ostomy bag device comprising a bag comprising a first chamber and a second chamber in communication via a first opening in a divider between the first chamber and the second chamber, the bag including a second opening configured to interface with the stoma exit and receiving waste in the first chamber, a third opening configured to expel the waste from the first chamber, and a fourth opening configured to release gas from the second chamber, the ostomy bag device comprising a first pressure sensitive interface located at the first opening configured to transition from a first state in which the first pressure sensitive interface prevents a flow of gas through the first opening from the first chamber to the second chamber and a second state in which the first pressure sensitive interface allows the flow of gas through the first opening from the first chamber to the second chamber, the ostomy bag device comprising a second pressure sensitive interface including a release valve located at the fourth opening configured to transition from the first state in which the release valve prevents a flow of gas through the fourth opening from the second chamber to an environment exterior to the bag and the second state in which the release valve allows the flow of gas through the fourth opening from the second chamber to the environment wherein the release valve comprises an actuator to manually open the release valve to allow the flow of gas through the fourth opening or manually close the release valve to prevent the flow of gas through the fourth opening; and
opening the release valve to allow the flow of gas through the fourth opening.

38. The method of claim 37, wherein a first pressure differential in the first chamber relative to the second chamber causes the first pressure sensitive interface to assume the first state preventing the flow of gas and a second pressure differential in the first chamber relative to the second chamber higher than the first pressure differential causes the first pressure sensitive interface to assume the second state allowing the flow of gas.

39. The method of claim 38, wherein the first pressure sensitive interface comprises a valve configured, in the first state, to block the first opening and, in the second state, to at least partially unblock the first opening.

40. The method of claim 39, wherein the valve comprises a spring attached to a first end of the valve and a second end of the valve, the first end of the valve being attached to an interior of the second chamber, expansion of the spring causing the second end of the valve to extend at least partially through the first opening to block the flow of gas through the first opening, contraction of the spring causing the second end to withdraw from the first opening to allow the flow of gas through the first opening.

41. The method of claim 40, wherein the first pressure differential allows the spring to expand and the second pressure differential forces the spring to contract.
